(19) United States
(12) Patent Application Publication
(10) Pub. No.: US 2016/0081713 A1
(43) Pub. Date: Mar. 24, 2016

(54) DEVICE FOR ACCESS TO PREFORMED BODY CAVITIES

(71) Applicants: Peter STOCKER, Knittelfeld (AT); Mario WIESER, Graz (AT)

(72) Inventors: Peter STOCKER, Knittelfeld (AT); Mario WIESER, Graz (AT)

(21) Appl. No.: 14/766,266

(22) PCT Filed: Jan. 16, 2014
(86) PCT No.: PCT/AT2014/050013

§ 371 Date: Nov. 24, 2015

(30) Foreign Application Priority Data
Feb. 7, 2013 (AT) A 50093/2013

(51) Int. Cl. A61B 17/34; A61B 17/3209

(52) U.S. Cl. CPC A61B 17/3415; A61B 17/32093; A61B 17/3417; A61B 2017/346; A61B 2017/3492

(57) ABSTRACT

The invention relates to a device (1) for access to preformed body cavities, such as thoracic drains, in particular for arthroscopy or laparoscopy on the human or animal body, comprising a base (3), a shaft (4) guided in the base (3), which shaft is designed to be longer than the base (3), and a cutting instrument (5), which is arranged in the shaft (4) and is guidable therein and which upon forward movement carries the shaft (4) as far as a predetermined position beyond the base (3). A device (1) according to the invention is characterised by ease of use, in particular for inexperienced surgeons, and the opportunity for rapid implementation of an arthroscopic procedure by means of a minimally invasive intervention in the body.

The invention relates to a device for access to preformed body cavities, such as thoracic drains, in particular for arthroscopy or laparoscopy on the human or animal body.

Today, arthroscopic procedures on the human or animal body belong to the standard methods of surgical medicine. In particular, arthroscopy on the human body, for example in the knee or shoulder region, represents a routine procedure. Each year, thousands of operations are performed on human knees, for example, in the case of the repair of cruciate ligament ruptures or damage to the meniscus.

Even though arthroscopy on the human knee is a routine procedure that can normally be performed relatively quickly and, in many cases, also only involving spinal amnesia or plexus amnesia of the region undergoing arthroscopy, complications can frequently occur. On the one hand, these complications concern the operation as such. On the other hand, an improper operation can also induce post-operative syndromes in the form of bleeding, infections, wound healing disturbances, dysfunction and pain. In addition, it must in particular also be considered that the introduction of bacteria, for example in the joint cavity of a human joint, after the operation can lead to serious inflammations of the joint or cavity that was operated on.

Today, arthroscopy on the human knee is normally performed in such a manner that the surgeon makes an incision in the skin using a scalpel. The subcutaneous regions of connective tissue layers underneath the skin must then also be cut through to facilitate subsequent penetration into the knee joint cavity. In addition to a layer of fat, firm connective tissue layers located thereunder must thereby also be cut through. Particularly in the case of the knee joint, it is necessary to partially sever fibers of what is referred to as the reserve stretching apparatus in order to enable penetration into the knee joint area. A severing of the fibers thereby usually also occurs transverse to the fiber direction, as a result of which the fibers are often damaged. If such an incision is made, a tube with a camera is then inserted in the region of the incision in order to perform an inspection of the joint. Here, it is often necessary to reposition the relevant extremity in order to be able to suitably inspect the entire joint area. However, the skin and the underlying regions can thereby move relative to one another, so that the incision made previously is no longer vertically aligned in the individual layers, but rather the individual incision regions are displaced relative to one another. In such a case, it is necessary to make an incision again, which is not desirable. If the camera is inserted into the knee joint, after multiple incisions as the case may be, and if possible damage to the joint apparatus is found, the arthroscopic instrument is introduced in a separate region, wherein an incision must be made again, as a result of which the same problems occur again, especially in the case of a repositioning. Once the damage that was found is repaired, the arthroscopic instrument and the camera are removed and the incisions are closed using suture material.

An operation as described above, for example to repair damage to the meniscus, typically lasts approximately 30 minutes if no complications occur in the performance of the operation. However, this applies to an experienced surgeon. For this type of surgeon, it would be even more efficient if the operating time could be shortened. For an inexperienced surgeon, the operating time can be considerably longer. In particular, creating access to the knee in a gentle manner often poses greater difficulty to an inexperienced surgeon.

In connection therewith, the object of the invention is to specify a device for arthroscopy with which bodily access can be created quickly and securely, for example for arthroscopy and laparoscopy or generally for a penetration into preformed body cavities of a human joint or other preformed body cavities.

The object is attained by a device for arthroscopy on the human or animal body, comprising a base, a shaft guided in the base, which shaft is embodied longer than the base, and a cutting instrument which is arranged in the shaft and is guidable therein and which, upon forward movement, carries the shaft as far as a predetermined position beyond the base.

A device according to the invention has the advantage that the base can be attached directly or indirectly to the human body and then serves as a stabilization for the shaft that is inserted or is to be inserted, in the interior of which the cutting instrument is smoothly guidable. By means of the corresponding arrangement of the individual components, only the fixing of the apparatus in a region of the body in which the operation is to take place is initially necessary. If the cutting instrument is driven forward, it makes the necessary incision in the human body to a desired depth. At the same time, the shaft is also carried as far as a predetermined position by the cutting instrument being driven forward, so that the shaft also penetrates into the human body while simultaneously enveloping the cutting instrument. In this manner, first the skin and then the subcutaneous regions connected thereto are cut through by the cutting instrument, and the surrounding shaft, which is carried into the body by the cutting instrument, spreads open the cut skin and the subsequent layers, so that perfect access is ensured for a subsequent operation. Once the cutting instrument with the shaft is inserted into the human body all the way to a desired point, the cutting instrument can be pulled out of the shaft. The base, together with the shaft, remains attached to the human body and then forms the access for the arthroscopic instrument that is to be inserted. Once the operation is complete, the arthroscopic instrument is first guided out of the shaft. The shaft is then removed from the base and, finally, the base is removed.

For a smooth guidance of the cutting instrument, it can be provided that the shaft comprises guides for the cutting instrument, which guides interact with corresponding structures of the cutting instrument. For example, the shaft can comprise guide grooves in which lateral extensions of the cutting instrument slide, which instrument is essentially embodied in an elongated manner. Also possible are other types of guides that allow a linear translation of the cutting instrument in the shaft without the cutting instrument rotating relative to the shaft.

In order for the cutting instrument to carry the shaft into the body, preferably at least one stop is provided in the shaft, which stop is ultimately struck by the cutting instrument after insertion into the shaft and continued forward movement in order to carry the shaft. The cutting instrument is then first inserted into the shaft and slides therein. Once the stop is reached, the cutting instrument carries the shaft upon continued forward movement. The position of the stop is thereby expediently chosen such that the shaft is carried as soon as the cutting instrument penetrates into the human body. The cutting instrument and shaft then penetrate into the human body virtually in unison, wherein a tip of the cutting instrument, which tip produces a cutting effect, is positioned slightly ahead the trailing shaft end in the direction of forward drive.

Particularly preferably, it is provided that the cutting instrument is embodied at a first end with at least one blade and is guidable in the shaft until the at least one blade protrudes past the shaft, preferably slightly protruding approximately in the region of a shaft end. This enables, in a particularly efficient manner, a combination of the incision that is to be made and the forward drive of the shaft for the purpose of creating access for an arthroscopic instrument.

At one end, the cutting instrument comprises at least one blade. However, multiple blades can also be provided. In order to not damage the fibers of the reserve stretching apparatus too severely during arthroscopy on a human knee, for example, and to thus avoid post-operative symptoms, the profile of the one cutting edge or the possibly multiple cutting edges lies on a single plane. It is then possible to position the cutting instrument such that an incision runs parallel to a fiber direction, whereby fiber damage can be minimized. The at least one blade can thereby be embodied conically and/or in a receding manner from a center to sides of the cutting instrument.

Adjacent to the cutting edge or cutting edges, a contour which extends in a conically widening manner from the cutting-active end of the cutting instrument to the opposite end thereof enables a gentle spreading-open of the cut region, which is subsequently spread open even farther by the shaft. If the cutting edge or the cutting edges are embodied in a receding manner towards the sides of the cutting instrument, that is, if they recede from a center of the cutting-active first end of the cutting instrument to the opposite second end thereof, a cutting pressure is particularly small. This has an advantageous effect on a preservation of the fiber structure. In addition, a slightly slower cutting occurs compared to cutting edges embodied in a straight manner, which is likewise advantageous. At the same time, a uniform cutting pressure and a particularly suitable centering are ensured.

Advantageously, the base is formed from an elastic material, in particular an elastically deformable plastic. The base serves as a stabilizing unit for the inserted shaft. However, it may still be necessary, after an incision is made and if the shaft is already positioned in the body, to deflect the shaft about its longitudinal axis. This is possible if the base is formed from an elastic material which allows a certain deflection or, within certain limits, a tipping of the shaft held therein.

The base can comprise one or multiple side walls which define a hollow space, wherein a first opening is provided in a head region of the base and a second opening is provided in an opposite region of the base. The shaft is then inserted in the region of the first opening in the head region and is held there by the base. Through the construction of the base from an elastic plastic, the desired angle variability for the shaft is also achieved when the shaft is positioned in the base. Here, a bead-shaped extension running around the first opening can be arranged in the head region of the base, in which at least one recess is embodied adjacently towards the second opening and the preferably transparent shaft is provided with one or multiple lateral bracing projections that engage in the at least one recess. The bracing projections preferably engage in the recesses precisely when, after the shaft is inserted into the body, the end position of the shaft is reached. Here, too, it is expedient that the base is formed from an elastically deformable material, since in this case the bead-shaped extension is initially pressed apart by a slight application of force, after which the bracing projections lock into place in the recess(es). For this purpose, the bracing projections are advantageously embodied in a wedge shape, so that a slight pressing-apart of the bead-shaped extension can occur.

From similar considerations stated above for the cutting instrument, the shaft is also embodied in a tapered manner at a first end and with a widening open cone at the opposite end. As a result of the tapered design at the first end, a wedge-shaped effect is achieved when the shaft is inserted into the body, which leads to a gentle spreading-open of the cut regions. The widening cone at the opposite end is provided on the one hand in order to only allow the shaft to be driven forward into the base as far as this cone. On the other hand, the funnel-shaped opening at the end of the shaft is expedient in order to be able to subsequently insert the arthroscopic instrument in an accurate manner, which is in particular an advantage for inexperienced surgeons.

Expediently, a guide pin is arranged on the cutting instrument at the bottom end. The guide pin serves to stabilize the cutting instrument.

It is not necessary, but provides a significant advantage, if a substrate adhesively attachable to the body is provided, onto which substrate the base is attached. In this case, the base does not bear against the body directly, but rather against the substrate that can be adhered to the body. A particularly simple positioning of the device is thus ensured. For this purpose, it can be provided in particular that the substrate is self-adhesive, so that the entire device can be adhered to the human body without additional means. The substrate is thereby preferably embodied in the shape of a small plate, that is, as thin as possible, and is connected to the base by a predetermined breaking point. The base arranged on top of the substrate, generally a membrane, then serves as a guide for the shaft and the cutting instrument guided therein, but also covers the substrate and thus acts as a barrier against germs. If the operation is complete and the shaft has already been removed from the base, the base can be released from the substrate or membrane by means of the predetermined breaking point. The membrane can then remain adhered to the human body in order to ensure a closure of the wound. In this case, the device is suitable not only for creating access for arthroscopy, but also ensures optimal wound care.

For arthroscopy that is camera-assisted, a set of two devices according to the invention is advantageous. The cutting instruments thereby each comprise one guide pin, wherein the guide pins have different lengths. A first device with the shorter guide pin is then provided for making an incision and for access for a camera. The second device with the longer guide pin serves to create access for an arthroscopic instrument that is to be inserted.

Additional features, advantages and effects of the invention follow from the exemplary embodiment described below. In the drawings which are thereby referenced:

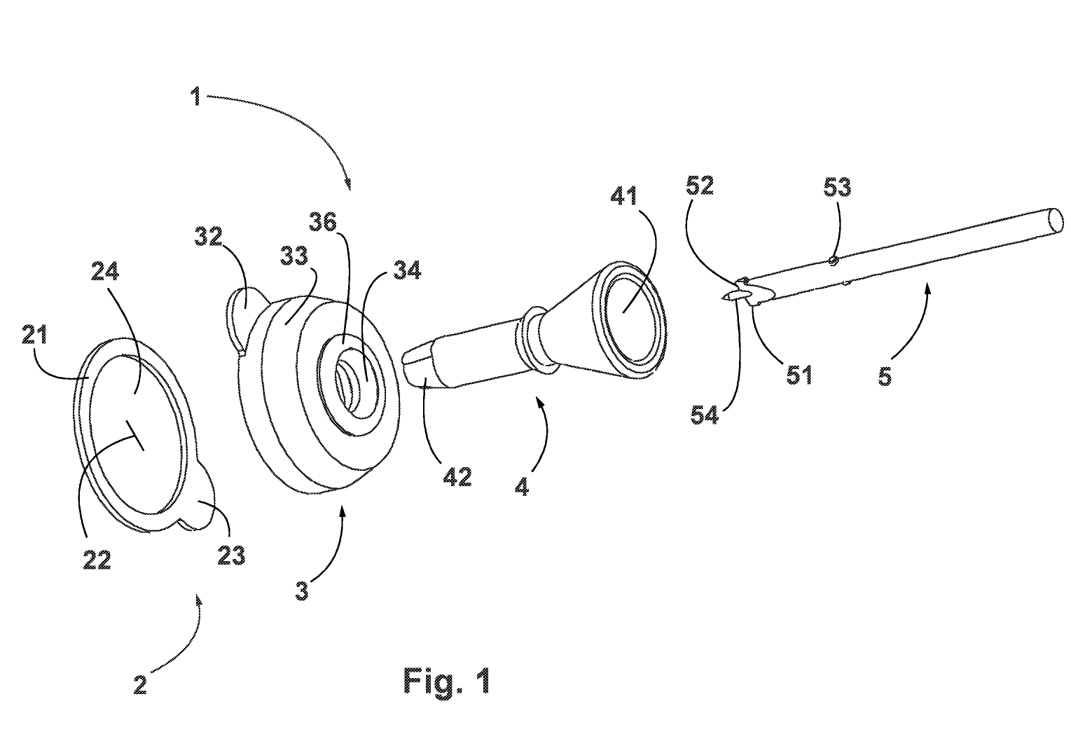

FIG. 1 shows an exploded illustration of a device according to the invention;

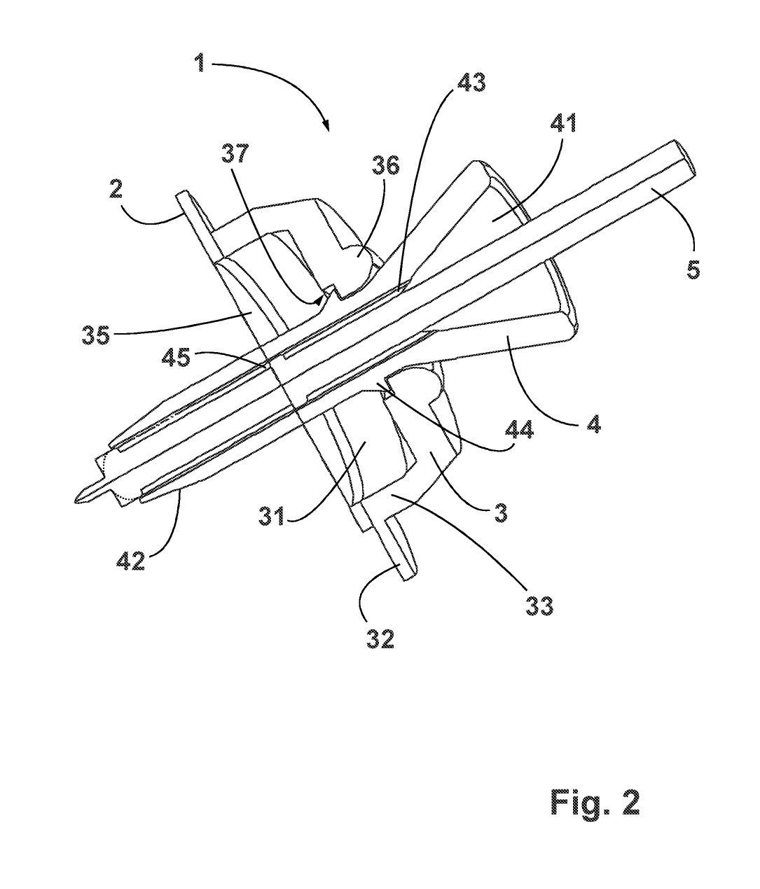

FIG. 2 shows a section through a device according to the invention;

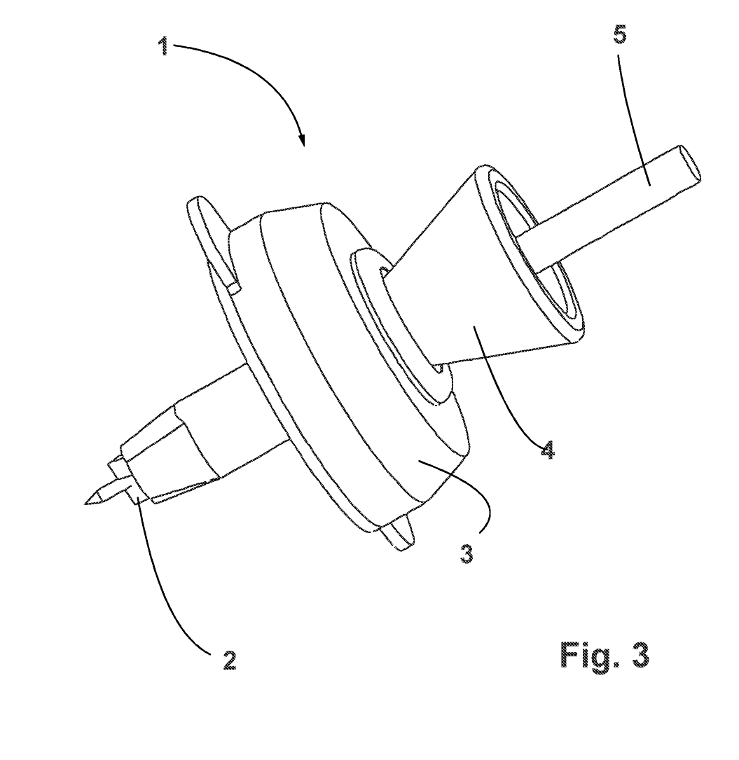

FIG. 3 shows a device according to the invention in a state in which it is assembled and inserted in a human body.

In FIG. 1, the individual parts of a device 1 according to the invention are illustrated in an exploded drawing. The device 1 according to the invention comprises a substrate 2, a base 3, a shaft 4 and a cutting instrument 5. These components, as well as their interaction, are explained below.

The substrate 2 is embodied as an easily penetrable membrane, wherein a central plastic film 24 is provided that is surrounded by a slightly stronger plastic ring 21. The membrane furthermore comprises a slit 22 into which the cutting instrument 5 explained below initially penetrates. The substrate 2 or the membrane is at a bottom end embodied in a self-adhesive manner, so that the membrane and thus also the other components can be easily affixed to a human body in order to subsequently create access for an arthroscopic instrument. The membrane primarily serves the purpose of forming a barrier against germs in the region of a subsequent incision. In addition, the substrate 2 comprises a tab 23 with which the substrate 2 can easily be released from the body again following the operation or possibly also after temporary wound care.

The base 3, the design of which can be seen particularly well in the sectional illustration according to FIG. 2, is arranged on the substrate 2 and connected thereto by a predetermined breaking point. The base 3 comprises a central hollow space 31 that is defined by a side wall 33. If necessary, the hollow space 31 can be flushed with a fluid. It is also possible to apply an excess pressure or negative pressure if this should be necessary for operation-related reasons. The base 3 has a certain height which, however, is smaller than a length of the shaft 4 that will be explained below. The base 3 comprises a first opening 34 in the head or cover region and a second opening 35 at the opposite end, the end adjacent to the membrane. The diameter of the first opening 34 is sized such that the shaft 4 can be accommodated in a precisely fitting manner. The second opening 35 is embodied considerably larger and essentially completely covers the underlying membrane or substrate 2. In the region of the end of the shaft 3 adjacent to the membrane, a tab 32 is provided. A bead-shaped extension 36 is positioned at the opposite end of the base 3, which extension ultimately defines the first opening 34. As can be seen in particular in FIG. 2, the bead-shaped extension 36 is slightly rounded at its head. A circumferential recess 37 is provided towards the hollow space 31, although multiple recesses 37 of this type can in principle also be present. However, it is expedient that a single recess 37 is provided which is, like the shaft 3, embodied in a round manner and extends around the opening region in the region of the first opening 34.

The shaft 4 mentioned above is embodied in an elongated and transparent manner. A length of the shaft 4 is significantly greater than a height of the base 3. It is expedient that the shaft 4 is at least twice as long as the height of the base 3. The shaft 4 comprises a first end that can be inserted into the body, which end is embodied in a conically widening manner or with a bottom cone 42 extending in the direction of an opposite end of the shaft 4. As a result, the shaft 4 gently spreads open first the skin and then the underlying tissue during the penetration into the human body. Adjacently, the shaft 4, which is embodied in a hollow manner, extends essentially cylindrically, wherein wedge-shaped bracing projections 44 are provided on an outer side. After the bracing projections 44, an open cone 41 is provided, by means of which the shaft 4 widens while forming a funnel-shaped inlet for other instruments. Optionally, two additional thin membranes not illustrated can also be provided in the shaft 4, which membranes must be cut through by the cutting instrument 5 during forward motion or insertion. These additional membranes in the shaft 4 serve as added barriers to germs and fluids. Furthermore, inside grooves 43 and at least one stop 45 are also provided in the interior.

Finally, the cutting instrument 5 is designed in a cylinder shape and can be embodied in a hollow or solid manner. The cutting instrument 5 comprises at an end 51 a guide pin 54 with a tip at its end. Adjacent to the guide pin 54, blades 52 extend which, as is illustrated, can run in a straight manner, but are preferably embodied in a receding manner towards the side of the cutting instrument 5, as this is indicated by dashed lines in FIG. 2, so that among other things a slight cutting pressure of the cutting instrument 5 is achieved with a stable guidance and so that fiber damage can be avoided to the greatest possible extent. Similar to the shaft 4 with which it interacts, the cutting instrument 5 is essentially embodied in an elongated manner, and it additionally comprises extensions 53 positioned on a side, which extensions interact with the stop 45 of the shaft 4. The extensions 53 are embodied slightly higher than the width of the blades 52 so that the blades 52 can slide past the stop 45 of the shaft 4. It is also possible that the extensions 36 are arranged on the cutting instrument 5 in a position skewed by 90° and that they slide in different grooves than the blades 52.

The functional principle of a device 1 according to the invention is explained for an arthroscopic procedure in reference to FIGS. 1 through 3. For the operation, the device 1 is already available as an assembled instrument. The base 3 is thereby attached to the substrate 2 and connected thereto by the predetermined breaking point. The shaft 4 is already held in the base 3, but only bears against the substrate 2 or membrane. The cutting instrument 5 is also already positioned in the shaft 4. If the cutting instrument 5 is then driven forward along its longitudinal axis, it first moves forward in a smoothly guided manner as a result of the guidance by the shaft 4 or the guidance of the extensions 53 in the inside grooves 43. Eventually, the cutting instrument 5 reaches the membrane and passes through it in the region of the slit 22. Shortly thereafter, the extensions 53 of the cutting instrument 5 strike the stops 45 of the shaft 4, as a result of which a continued forward movement of the cutting instrument 5 causes a carrying or transport of the shaft 4. This is then the case when the blades 52 of the cutting instrument 5 slightly protrude past the bottom cone 42 of the shaft 4. To allow the shaft 4 to easily slide into the skin, the blades 52 are for this purpose embodied roughly as wide as an end of the shaft 4. The cutting instrument 5, together with the shaft 4, is then driven forward into the body until the position illustrated in FIG. 2 or 3 has approximately been reached. The bracing projections 44 of the shaft 4 then bear against the bead-shaped extension 36 of the base 3. Because the base 3 is made of an elastic material, its resistance can be overcome by a slight application of force, so that the bracing projections 44 slide over the bead-shaped extension 36 and ultimately come to rest in the recesses 37 of the base 3. As a result, the shaft 4 is perfectly secured against an unintended loosening from the base 3 during the further course of the operation. Aside from this, the elastic deformability of the base 3 also allows the shaft 4 to deflect angularly during the operation. Simultaneously with the fixing of the bracing projections 44, the open bottom cone 42 strikes the bead-shaped extension 36, so that the shaft 4 is essentially secured against a further displacement in both directions. The cutting instrument 5 also cannot be moved any farther. The situation illustrated in FIG. 2 or 3 is now achieved. The cutting instrument 5 is then pulled out of the shaft 4. In this manner, access is created to the body that is connected to the substrate 2 or membrane. A camera or, if necessary, an arthroscopic instrument can then be inserted. If both components are necessary during the operation, that is, both the camera and also the arthroscopic instrument, the surgeon proceeds at two different locations as described above. Following the arthroscopic procedure, the arthroscopic instrument or camera is pulled out of the shaft 4. The shaft 4 is then released from the base 3 in that the shaft is pulled against the bracing force produced by the bracing projections 44 in combination with the recess 37. Because of the elastic deformability of the base 3, this is possible with a moderate application of force. Only the base 3 is then located on the membrane. The tab 32 is used to pull the base 3 off from the membrane. The base 3 can be released from the membrane as a result of the connection via a predetermined breaking point. The substrate 2 or membrane can also be removed using its tab 23. However, it is advantageous if the membrane continues to remain on the body to dress the incision or wound until the wound has fully healed. Because the membrane only comprises a small slit and is also embodied elastically in the central region, it essentially closes over the wound, which enables a very good healing and, most importantly, protects the open wound against germs.

1. Device (1) for access to preformed body cavities, such as thoracic drains, in particular for arthroscopy or laparoscopy on the human or animal body, comprising a base (3), a shaft (4) guided in the base (3), which shaft is embodied longer than the base (3), and a cutting instrument (5) which is arranged in the shaft (4) and is guidable therein and which, upon forward movement, carries the shaft (4) as far as a predetermined position beyond the base (3).

2. Device (1) according to claim 1, wherein the shaft (4) comprises guides for the cutting instrument (5), which guides interact with corresponding structures of the cutting instrument (5).

3. Device (1) according to claim 1, wherein the shaft (4) comprises guide grooves (44) in which lateral extensions (53) of the cutting instrument (5) slide.

4. Device (1) according to claim 1, wherein at least one stop (45) is provided in the shaft (4), which stop is struck by the cutting instrument (5) after insertion into the shaft (4) and forward movement in order to carry the shaft (4).

5. Device (1) according to claim 1, wherein the cutting instrument (5) is embodied at a first end (51) with at least one blade (52) and is guidable in the shaft (4) until the at least one blade (52) protrudes past the shaft (4), preferably slightly protruding approximately in the region of a shaft end (46).

6. Device (1) according to claim 5, wherein the at least one blade (52) is embodied conically and/or in a receding manner from a center to sides of the cutting instrument (5).

7. Device (1) according to claim 1, wherein the base (3) is formed from an elastic material, in particular an elastically deformable plastic.

8. Device (1) according to claim 1, wherein the base (3) comprises one or multiple side walls (33) which define a hollow space (31) and wherein a first opening (34) is provided in a head region and a second opening (35) in an opposite region of the base (3).

9. Device (1) according to claim 1, wherein a bead-shaped extension (36) running around the first opening (34) is arranged in the head region of the base (3), in which at least one recess (37) is embodied towards the second opening (35) and the shaft (4) is provided with one or multiple lateral bracing projections (44) that engage in the at least one recess (37).

10. Device (1) according to claim 1, wherein the shaft (4) is embodied in a tapered manner at a first end and with a widening open cone (41) at the opposite end.

11. Device (1) according to claim 1, wherein a guide pin (54) is arranged on the cutting instrument (5) at the bottom end.

12. Device (1) according to claim 1, wherein a substrate (2) adhesively attachable to the body is provided, onto which substrate the base (3) is attached.

13. Device (1) according to claim 12, wherein the substrate (2) is self-adhesive.

14. Device (1) according to claim 12, wherein the substrate (2) is embodied in the shape of a small plate and connected to the base (3) by a predetermined breaking point.

15. Set of multiple devices (1) according to claim 1, wherein the cutting instruments (5) each comprise one guide pin (54), wherein the guide pins (54) have different lengths.
